(19) United States
(12) Patent Application Publication
(10) Pub. No.: US 2010/0286547 A1
(43) Pub. Date: Nov. 11, 2010

(54) BREATHING DETECTION DEVICE AND METHOD FOR DETECTING ASYNCHRONOUS OR ARRESTED BREATHING

(76) Inventor: Ahmed AOUDE, Montreal (CA)

Correspondence Address: Andrew W. Chu, PC, P. O. Box 2925, Houston, TX, 77252-2925, US

(21) Appl. No.: 12/774,598

(22) Filed: May 5, 2010

(30) Foreign Application Priority Data
May 6, 2009 (CA) 2665651

(51) Int. Cl. A61B 5/08

(52) U.S. Cl. 600/534; 600/529

(57) ABSTRACT

The present invention in one aspect is a breathing detection device with a signal source and receiver. A card between the source and receiver alternates between a first state of signal reception and a second state of signal interference, on card movement. A cord moves the card in one direction on pulling, and in reverse on retraction. The card can be a disc, belt, or any configuration conforming to a principle of card movement to affect a difference in signal receiving state. Another aspect the invention is an asynchronous breathing detection method providing a first and a second detection device as described, both in communication. In various configurations, one or more processors is provided to receive and process data to determine magnitude and direction of cord and card movement.

### RELATED U.S. APPLICATIONS

Not applicable.

### STATEMENT REGARDING FEDERALLY SPONSORED RESEARCH OR DEVELOPMENT

Not applicable.

### REFERENCE TO MICROFICHE APPENDIX

Not applicable.

### BACKGROUND OF THE INVENTION

1. Field of the Invention

The present invention is in the field of apnea, and more specifically is a device to detect apnea and a method for detecting asynchronous breathing and breathing arrest.

2. Description of Related Art Including Information Disclosed Under 37 CFR 1.97 and 37 CFR 1.98

Apnea/Apnoea/Apnœa is a technical term for temporary suspension of external breathing, during which there is no movement of respiratory muscles and lung volume initially remains unchanged. Depending on airway patency, there may not be gas flow between lungs and external environment; gas exchange within the lungs and cellular respiration is not affected. Sleep apnea means temporary suspension of regular breathing during sleep. Sleep apnea can be fatal especially in infants and the elderly.

Sleep apnea or breathing arrest detection is important for both diagnosis and treatment, and can be detected by various methods.

One method is to visually monitor a person while asleep, and note when breathing stops. Visual monitoring is time consuming, and requires constant attention.

Another method uses nasal strips with microprocessor implants (like SLEEPSTRIP™, trademark of SLP Scientific Laboratory Products Ltd.). The implants measure the number of times breathing stops and then determines after a first night, sleep apnea severity. This is problematic because a) sleep breathing data is trapped within the nasal strip, inaccessible to an outside observer; b) the strips' microprocessors are limited, used once and disposed of; and c) attaching a strip to one's nose can be uncomfortable, and interfere with sleep.

Another method is respiratory inductance plethysmography (RIP). RIP is an automated breathing detection method using electric coils. It has good breathing detection ability, but yields high false positives when used for apnea detection. Slight movement during sleep can disrupt detection accuracy. RIP measures breathing by directly translating movement, as opposed to forming a calculation from a data set. RIP is complex and unreliable.

### SUMMARY OF THE INVENTION

The present invention in one aspect provides a breathing detection device having a signal source, to emit a signal, and a signal receiver, to receive the signal. A card is disposed between the source and receiver to alternate between a first state of receiver signal reception and a second state of receiver signal interference, upon movement of the card. A movement means to move the card is provided. A cord defining two ends, is provided with the first end being attached to the movement means to move the card in one direction on pulling of the cord, and in a reverse direction on retraction of the cord. In one embodiment the card is a disc, and in another is a belt. Card type can vary.

Another aspect of the invention is an asynchronous breathing detection method providing a first and a second breathing detection device as described above, both in communication. A processor is provided in communication with the first and second device, to receive and process data.

### BRIEF DESCRIPTION OF THE DRAWINGS

FIG. 1 is a perspective view of a breathing detection device encased in a housing.

FIG. 2 is a schematic view of a breathing detection device.

FIG. 3 is a perspective view of an encoder inside a breathing detection device, in a disc-axle-cogwheel configuration.

FIG. 4 is a perspective view of another encoder inside a breathing detection device, in a belt-conveyor-cogwheel configuration.

FIG. 5 is a mixed plan-perspective view of two detection devices in communication, embedded or attached to clothing, to detect asynchronous breathing.

### DETAILED DESCRIPTION OF THE DRAWINGS

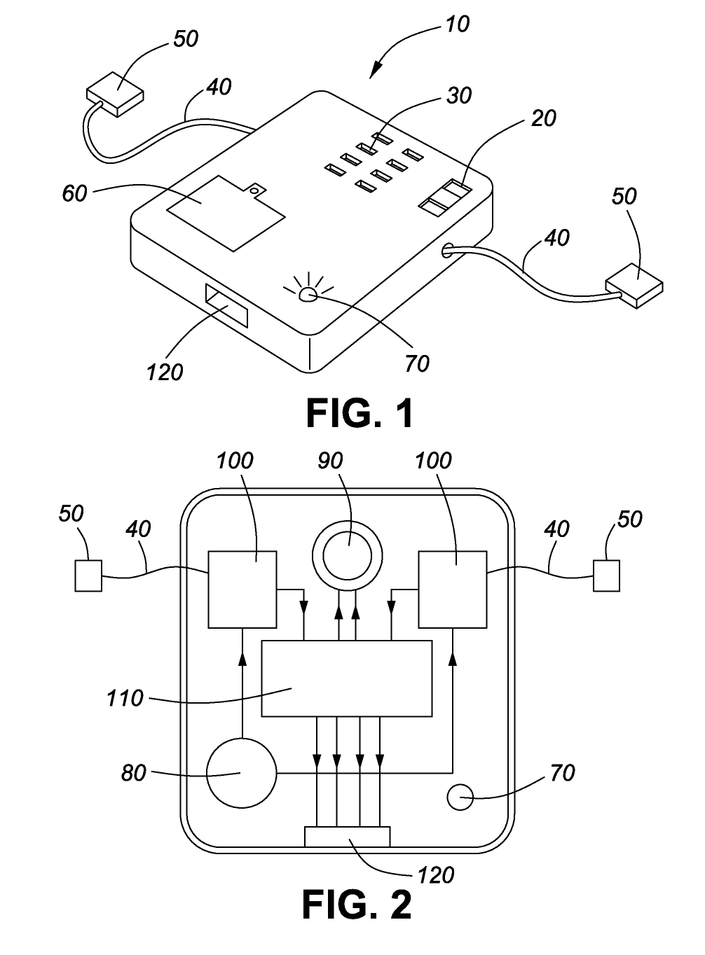

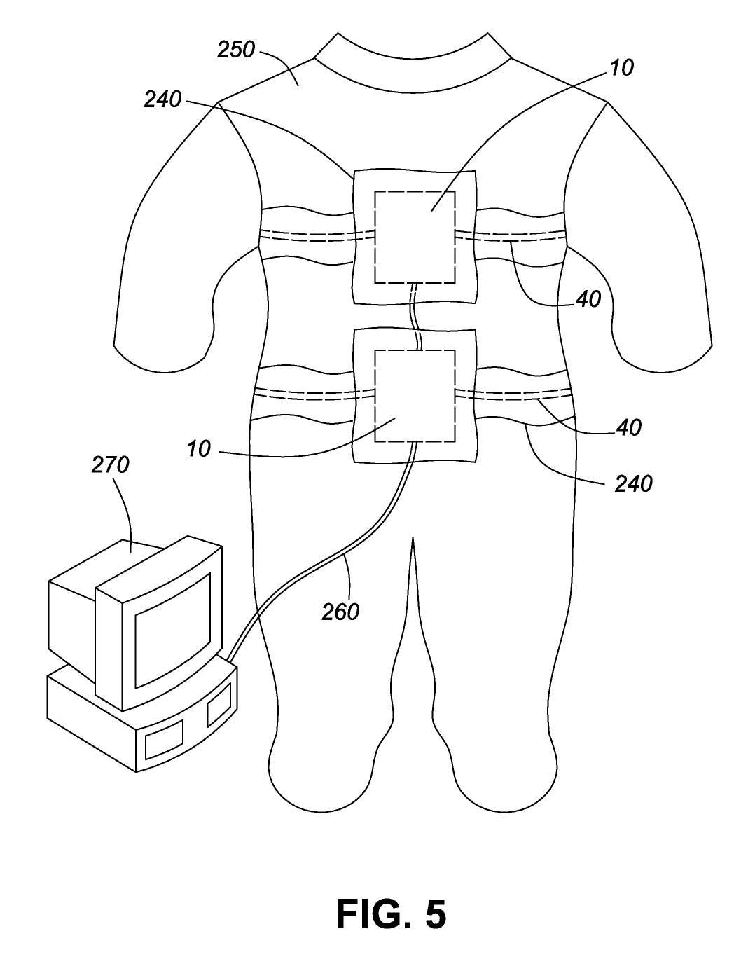

FIG. 1 shows a breathing detection device encased in housing (10) generally, in accordance with a first aspect of the present invention. The housing (10) provides for practical carrying of the present invention, along with useful features like an on/off switch (20) to indicate on/off status, and a speaker vent (30) for transmitting audible alarms and data through a speaker (90). If so-powered, a battery access panel (60) can be provided to access a battery (80), as well as a light or visual indicator (70) to indicate on/off state, and/or transmit visual data. Cords (40) enter the housing (10) for connection to encoders (100). The cords (40) have connectors for wrapping around, and connection to, a chest or abdominal area (FIG. 5). An access port (120) can be provided to accept cables (260), to do any of transmit, receive, and pass, data from encoders (100) to an outside processor (270) (meaning outside of the housing (10)) or any type of processors that usefully processes encoder (100) data, or to each other. An internal processor (110) (meaning inside the housing (10)) can be provided to process encoder (100) data within the device itself, as opposed to forwarding its data to an outside processor (270).

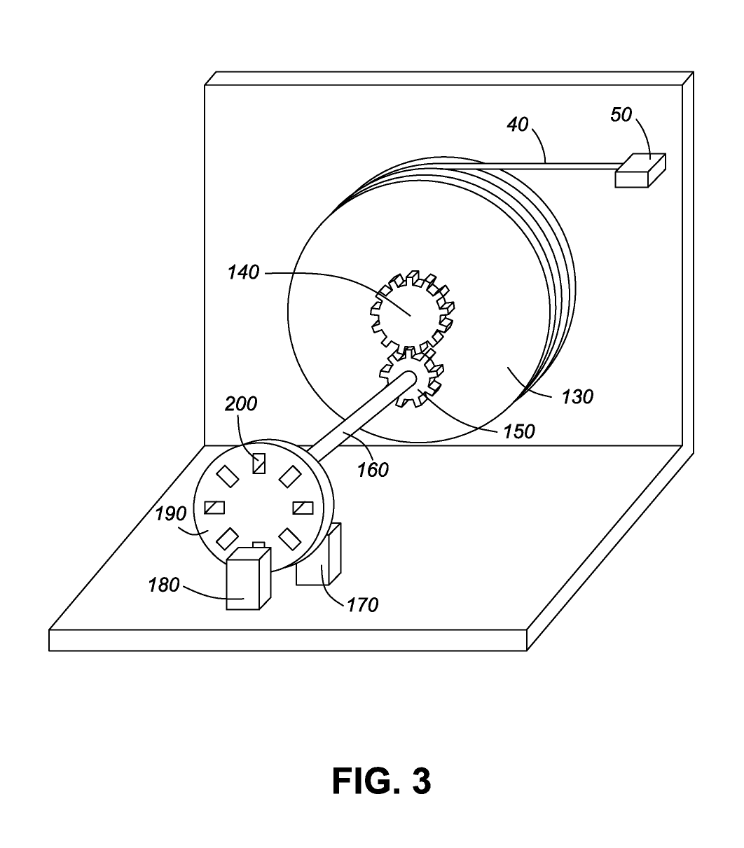

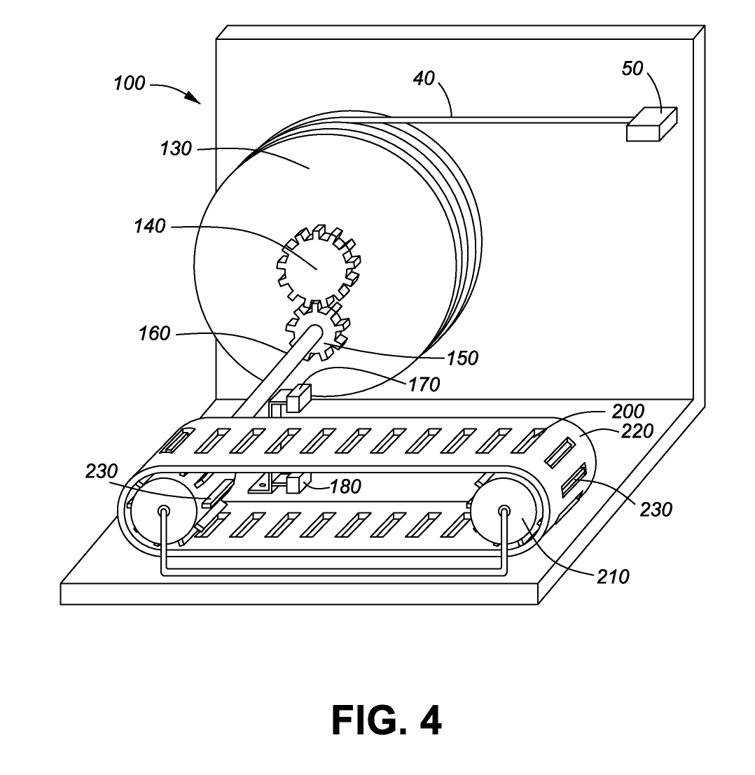

In one specific embodiment, an encoder (100) can be in a disc-axle-cogwheel configuration (FIG. 3). In this configuration, a signal source (170) to transmit a signal, and a signal receiver (180) to receive that signal, are provided. The signal can be of any type, including a light signal. A card is provided, in disc (190) or belt (220) form, or in any form conforming to a principle of card movement to affect a difference in signal receiving state (i.e. a binary data stream). In FIG. 3, the disc (190) is used as an example, defining multiple slot openings (200) through which a signal can pass without interference when placed between a source (170) and a receiver (180). The non-slot portions of the disc (190) alternate in a pattern with the openings (200), and interfere with receiver (180) signal reception when placed between a source (170) and a receiver (180). The alternating pattern, on disc (190) movement, reflects movement of the cord (40) (which can be pulled in one direction and retracted in a reverse direction (by a bias or spring (not shown))), which in turn reflects breathing movement (chest expansion and contraction, and abdominal expansion and contraction, both not shown). The card can be made of any material so long as it is suitable to alternately block and allow signal reception, and move as desired.

The cord (40) can be fed through a guide in the form of a spool (130), and engage a first cogwheel (140). On pulling of the cord (40) (chest or abdomen expansion (inhaling)) the first cogwheel (140) engages a second cogwheel (150). On chest or abdomen contraction (exhaling), the cord (40) is retracted by a bias or spring (not shown). An axle (160) extends from the second cogwheel and attaches to the disc (190). When the axle (160) rotates, the disc (190) moves between a first state of receiver signal reception and a second state of receiver signal interference. When the slot opening (200) is between the source (170) and the receiver (180), signals are free to pass; otherwise signals are blocked. On card movement, the alternating opening pattern produces a binary data stream useful to detect sleep apnea by computation. In this specific case interference means the signal from the source (170) is blocked completely. Alternating embodiments can provide slot openings (200) with various filters or sensors, to modify or amplify signal data, and interfere with signals in a way other than by blockage.

A different embodiment, similar in principle, is a belt-conveyor-cogwheel configuration. Instead of the axle (160) engaging a disc (190), it engages a conveyor (210). The conveyor (210) defines teeth (230) to engage slot openings (200) and move a belt (220) on axle (160) movement. The slot openings (200) alternately allow a signal to travel from the source (170) to the receiver (180) when placed there between; otherwise the signal is blocked. The change between the two states of signal reception identify and correspond with any of chest expansion and contraction, and abdominal expansion and contraction.

Both of the above and all embodiments conforming to a principle of card movement to effect a difference in signal receiving state, are not affected by accidental movement during sleep (unlike RIP).

These devices can be attached to or embedded in clothing (250), and can use padding (240) to ease any discomfort in wearing the detection device housing (10). An unexpected useful benefit is that these encoders (100) can be connected in communication, to wrap around a chest and abdomen respectively (FIG. 5). Data from both encoders (100) can be transmitted to an inside processor (110) or outside processor (270) by cable (260) or wireless transmission (not shown). Data analyzed from both encoders (100) can detect asynchronous breathing or breathing arrest.

A signal phase difference estimate can be computed by dividing a time the cords (40) are moving in opposite direction by a time period for a single breath. Mathematically this is Θ=(t/T), where Θ is a phase difference estimate, t is a time the cords (40) are moving in opposite direction, and T is a time for a single breath. The estimate can be compared to a threshold to decide if asynchronous breathing is present.

1. A breathing detection device comprising:
a signal source, to emit a signal;
a signal receiver, to receive the signal;
a card disposed between the source and the receiver to alternate between a first state of receiver signal reception and a second state of receiver signal interference, on movement of the card;
a movement means to move the card; and
a cord defining two ends, a first end engaging the movement means to move the card in one direction on pulling of the cord, and in a reverse direction on retraction of the cord.

2. The device in claim 1 further comprising a housing, the device being encompassed within the housing.

3. The device in claim 1 wherein the movement means is an axle and cogwheel configuration.

4. The device in claim 1 further comprising the cord guide attached to the movement means.

5. The device in claim 1 wherein the card is disc-shaped.

6. The device in claim 1 wherein the movement means comprises a conveyor adapted to receive a conveyor belt.

7. The device in claim 1 wherein the card is comprised of a conveyor belt.

8. The device in claim 1 further comprising an outside processor to process receiver data.

9. The device in claim 1 further comprising the power source to power the signal source.

10. The device in claim 1 further comprising a speaker to emit sound related to data received from the receiver.

11. The device in claim 1 further comprising a light-emitting diode to indicate on and off state.

12. The device in claim 1 further comprising an alarm to provide alerts related to receiver data.

13. The device in claim 1 further comprising an input and output to receive and transmit data, the input being in communication with the processor.

14. An asynchronous breathing detection method comprising the following steps:
providing a first breathing detection device comprising a signal source, to emit a signal; a signal receiver, to receive the signal; a card disposed between the source and receiver to alternate between a first state of receiver signal reception and a second state of receiver signal interference, on movement of the card; a movement means to move the card; and a cord defining two ends, the first end engaging the movement means to move the card in one direction on pulling of the cord, and in a reverse direction on retraction of the cord;
providing a second breathing detection device same as the first; the first and second devices being in communication; and
providing a processor in communication with the first and second device, to receive and process data to determine magnitude and direction of cord and card movement.

15. The method in claim 14 wherein the first and second detection devices are attached to clothing.

16. The method in claim 14 wherein the first and second detection devices are embedded in clothing.

17. The method in claim 14 wherein the first and second devices are connected about a chest and an abdomen respectively.

18. The device in claim 1 wherein the device is embedded in clothing.

19. The device in claim 1 further comprising at least one processor in communication with the receiver, to process data to determine magnitude and direction of cord and card movement.

20. The device in claim 1 further comprising at least one processor in communication with the receiver, to detect breathing arrest.

21. The device in claim 1 further comprising at least one processor to process signal phase difference.

22. A method for detecting asynchronous breathing comprising the following steps:
providing a first breathing detection device comprising a signal source, to emit a signal; a signal receiver, to receive the signal; a card disposed between the source and receiver to alternate between a first state of receiver signal reception and a second state of receiver signal interference, on movement of the card; a movement means to move the card; and a cord defining two ends, the first end engaging the movement means to move the card in one direction on pulling of the cord, and in a reverse direction on retraction of the cord;
providing a second breathing detection device same as the first; the first and second devices being in communication; and
using at least one processor in communication with the receivers, to process signal phase difference.

23. The method in claim 22 for use to detect breathing arrest.

24. The method in claim 22 for use to detect breathing asynchrony.

25. The method in claim 22 for use to detect asynchronous apnea.

26. The method in claim 22 for use to detect apnea
